(19) United States
(12) Patent Application Publication
(10) Pub. No.: US 2025/0098649 A1
(43) Pub. Date: Mar. 27, 2025

(54) METHODS FOR REDUCING INHERITED BOVINE CONGESTIVE HEART FAILURE RISK FACTORS IN A BOVINE POPULATION

(71) Applicant: J.R. SIMPLOT COMPANY, Boise, ID (US)

(72) Inventors: Randall RAYMOND, Boise, ID (US); Justin BUCHANAN, Boise, ID (US)

(21) Appl. No.: 18/728,254

(22) PCT Filed: Jan. 13, 2023
(86) PCT No.: PCT/US2023/060634

§ 371 Date: Jul. 11, 2024

Related U.S. Application Data
(60) Provisional application No. 63/299,384, filed on Jan. 13, 2022.

(51) Int. Cl. A01K 67/02; C12N 15/10; G16B 20/20; G16H 50/30

(52) U.S. Cl. CPC A01K 67/02; C12N 15/1089; G16B 20/20; G16H 50/30

(57) ABSTRACT

The disclosure relates to quantitative trait loci associated with BCHF. The disclosure further teaches methods of generating EPDs using a genotype-phenotype library and associated algorithms with individual genomic information. Methods of using the EPDs for breeding selection to reduce inherited risk factors for BCHF in a bovine population as well management of individuals who are at risk for BCHF are also included.

### CROSS-REFERENCE TO RELATED APPLICATION

This application claims the benefit of priority to U.S. Provisional Application No. 63/299,384, filed Jan. 13, 2022, the entire contents of which is incorporated herein by reference.

### TECHNICAL FIELD

This disclosure relates to quantitative trait loci (QTL) associated with Bovine Congestive Heart Failure (BCHF) and methods of using these genotype-phenotype associations to generate expected progeny differences for assisted selection of bovine seedstock to reduce inherited risk factors for BCHF in a bovine population.

### BACKGROUND

BCHF is a growing problem in the cattle industry, and a significant cause of death. BCHF is a condition that involves pulmonary hypertension that can lead to right ventricular failure. Some cattle operations are experiencing annual losses greater than $250,000. Thus, there remains a need in the industry for effective genetic screening measures to reduce BCHF incidence in future generations.

### SUMMARY

In some embodiments, the disclosure teaches a method for producing a bovine progeny, comprising selecting at least one male bovine having EPD criteria associated therewith, wherein the EPD criteria includes an EPD for BCHF, and wherein the BCHF EPD is less than 0.1, pairing the selected male with at least one female, and producing at least one bovine progeny.

In some embodiments, the disclosure teaches a method for producing an EPD that predicts the risk of BCHF in a progeny of at least one bovine animal, comprising obtaining genomic sequencing or SNP array data from the at least one bovine animal, analyzing the sequencing data or SNP array in conjunction with a phenotype-genotype library, wherein the phenotype-genotype library comprises a plurality of phenotypic heart and genomic variation data, and producing a BCHF EPD for the at least one bovine animal.

In some embodiments, the disclosure teaches a method of producing an expected progeny difference (EPD) algorithm, comprising utilizing a genomic best linear unbiased prediction (BLUP) model to determine a correlation between genetic variations present in a digital genomic library and a phenotypic metric present in a digital phenotype library to thereby produce an algorithm that calculates an EPD.

In some embodiments, the disclosure relates to a processor-readable non-transitory medium storing code representing instructions to be executed by a processor, the code comprising code to cause the processor to: (a) access data from a digital genomic library comprising genetic variations associated with each of a plurality of bovine animals; (b) access data from a digital phenotype library comprising phenotypic metrics associated with each of a plurality of bovine animals; (c) determine, utilizing a genomic BLUP model, a correlation between the genetic variations present in the digital genomic library and a phenotypic metric present in the digital phenotype library.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIGS. 1A-1E are photographs of hearts dissected from the kill floor and scored for congestive heart failure. FIG. 1A is a grade 1 normal heart. FIG. 1B is a grade 2 heart showing mild changes. FIG. 1C is a grade 3 heart showing moderate changes. FIG. 1D is a grade 4 heart showing severe changes. FIG. 1E is a grade 5 heart showing severe changes with flaccid (scale adopted from Holt, T. 2020. Heart visual grading image. Colorado State University).

FIGS. 2A-2D are Manhattan (FIGS. 8A and 8B) and q-q plots (FIGS. 8C and 8D) of McNemar's p-values for 1M genome-wide SNPs.

FIG. 3A-3D show data from PCA score plots (colors correspond to different cattle breeds). FIG. 3A compares PC1 with PC2, overlaid with BCHF incidence (FIG. 3B), while FIGS. 3C and 3D compare PC2 with PC3 and PC3 with PC4, respectively.

FIG. 4 is a box plot diagram showing the heart score associated with percent Angus ancestry.

FIGS. 5A-5D are logistic regression models of the probability (or risk) of a heart condition with Charolais ancestry (FIG. 5A), Angus ancestry (FIG. 5B), Hereford ancestry (FIG. 5C), and Holstein ancestry (FIG. 5D).

FIGS. 6A-6D show data related to carcass weight. FIG. 6A is a Manhattan plot, FIGS. 6B and 6C are q-q plots, and FIG. 6D is a histogram.

FIG. 7 is a flow diagram and related equation of EPD accuracy.

FIG. 8 is a histogram of EPD frequency in cattle.

FIG. 9 is a scatterplot of EPDs and associated heart scores within the individual.

### DETAILED DESCRIPTION

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

Following long-standing patent law convention, the terms “a,” “an,” and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of +10% from the specified amount, as such variations are appropriate to perform the disclosed methods and/or employ the disclosed compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one or more of the elements to which the “and/or” refers can also individually be present in single or multiple occurrences in the combinations(s) and/or subcombination(s).

As used herein, the term “sequence identity” refers to the presence of identical nucleotides or amino acids at corresponding positions of two sequences. Readily available sequence comparison and multiple sequence alignment algorithms are, respectively, the Basic Local Alignment Search Tool (BLAST®) and ClustalW/ClustalW2/Clustal Omega programs available on the Internet (e.g., the website of the EMBL-EBI). Some alignment programs are MacVector (Oxford Molecular Ltd, Oxford, U.K.) and ALIGN Plus (Scientific and Educational Software, Pennsylvania). Other non-limiting alignment programs include Sequencher (Gene Codes, Ann Arbor, Michigan), AlignX, and Vector NTI (Invitrogen, Carlsbad, CA). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001.

As used herein, “heritability” (h²) relates to the proportion of trait variance that is due to additive genetic factors.

As used herein, “seedstock” refers to cattle that are used for breeding. They have documented pedigrees and may also have estimates of genetic merit (such as expected progeny differences).

As used herein, “expected progeny differences” or “EPD” refers to the genetic component of an animal's phenotype that is expected to be passed on to the next generation.

As used herein, “high-density” refers to a genetic test with greater than or equal to, 100,000 markers.

As used herein, “low-density” refers to a genetic test with less than 100,000 markers.

“Quantitative trait loci” or “QTL”-A quantitative trait is one that has measurable phenotypic variation owing to genetic and/or environmental influences. This variation can consist of discrete values. A QTL is a genetic locus, the alleles of which affect this variation. Generally, quantitative traits are multifactorial and are influenced by several polymorphic genes and environmental conditions, so one or many QTLs can influence a trait or a phenotype.

### Overview

The disclosure relates to quantitative trait loci associated with BCHF. The disclosure further teaches methods of generating EPDs using a genotype-phenotype library and associated algorithms with individual genomic information. Methods of using the EPDs for breeding selection to reduce inherited risk factors for BCHF in a bovine population as well as management of individuals who are at risk for BCHF are also included.

### Bovine Congestive Heart Failure

BCHF, also known as “Feedlot heart disease” (FHD) or simply “congestive heart failure” (CHF), has historically been an issue in feedlot and seedstock cattle raised at higher altitudes. The increase in pulmonary arterial pressure (PAP) and extra fat leads to pulmonary hypertension (PH)—right-sided heart failure from increased pulmonary vessel pressures and cardiopulmonary remodeling. The remodeling of the heart can be observed and measured on a scale of 1 to 5, with a score of 1 being a normal heart and a score of 5 being a heart with extreme structural changes (Heffernan K., et al., Phenotypic relationships between heart score and feed efficiency, carcass, and pulmonary arterial pressure traits, Transl. Anim. Sci. 2020.4: S103-S107; Neary J. M., et al., Mean pulmonary arterial pressures in Angus steers increase from cow-calf to feedlot-finishing phases, J. Anim. Sci. 2015.93:3854-3861).

Prior to cardiac failure, signs of BCHF include syncope (a temporary loss of consciousness caused by a fall in blood pressure), weakness, intolerance to exercise, jugular vein distension and pulsation, subcutaneous edema, cardiac arrhythmias, cardiac murmurs, and muffled heart sounds (Raouf M., et al., Congestive heart failure in cattle; etiology, clinical, and ultrasonographic findings in 67 cases, Vet World. 2020.13 (6): 1145-1152). The prognosis of such conditions is mostly poor and the animals are advised to be slaughtered or euthanized (Braun U, et al., Clinical findings in 28 cattle with traumatic pericarditis. Vet. Rec. 2007; 161 (16): 558-563). Premature death and less efficient cattle leads to significant monetary loss. Some cattle operations are experiencing annual losses greater than $250,000.

The incidence of BCHF has doubled since the early 2000s. Some operations have reported as many as 20% to 35% of cattle showing some form of heart failure at harvest, some of which had never experienced high altitudes. Recently, a positive correlation between heart score and heart fat score was identified, and it is hypothesized that hearts having more fat deposits would have greater difficulty pumping efficiently (Kukor, I. M., et al., Sire differences within heart and heart fat score in beef cattle, Transl. Anim. Sci. 2021.5: S149-S153).

Two genes were also recently linked to an increased risk of BCHF, the arresting domain-containing 3 protein (ARRDC3) and nuclear factor IA (NFIA). Animals comprising specific SNPs in these genes were approximately 7- and 15-fold more likely to have BCHF (Heaton, M. et al., Association of ARRDC3 and NF1A genes with bovine congestive heart failure. In: Plant and Animal Genome Conference Proceedings, Jan. 13, 2020, San Diego, California, poster PE0326; Heaton, M. “Genetic association with bovine congestive heart failure (BCHF) in Feedlot cattle” In: Plant and Animal Genome Conference Proceedings, Jan. 13, 2020, San Diego, California, slide presentation). Thus, there may be additional underlying genetic components to BCHF that have yet to be investigated.

### Data Collection and BCHF Scoring

Observations of cattle heart scores were carried out on 32,763 individual animals from May 2020 to August 2022 by trained personnel at CS Beef Packers in Kuna, Idaho. All animals were shipped from the Simplot Land and Livestock feedlot located in Grand View, Idaho. Individual animal data including origin, weights, medications, breed appearance, and feed intake data was captured upon feedlot entry and throughout the feeding period with the use of electronic ID tags. All data was captured and stored in the Simplot database systems specific to the Land and Livestock division.

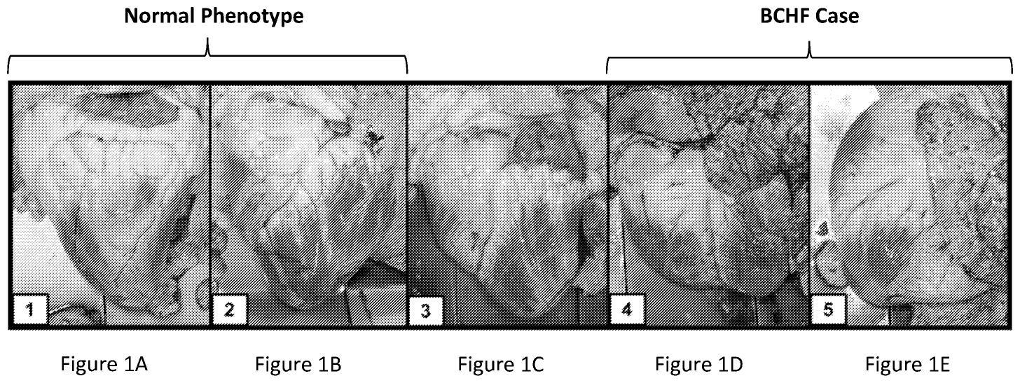

Heart scoring at CS Beef Packers was carried out utilizing a 1 to 5 visual scoring system developed at Colorado State University (available on the world wide web at academic.oup.com/tas/article/4/Supplement_1/S103/6043951) (FIGS. 1A-1E). Hearts were visually scored by trained personnel directly after the viscera were removed from each animal. In addition, carcass information including hot carcass weight, ribeye area, backfat thickness, and marbling score were collected on each carcass.

The heart scoring system was used to create a “normal” and a “case” definition for each phenotype used in the final analysis. Heart scores 1 and 2 were assigned to a normal phenotype definition, and heart scores 4 and 5 were assigned to a case phenotype definition. All heart scores of 3 were discarded from the analysis to increase specificity and reduce error that might be present due to scoring bias. As shown below in Table 1, out of the 17,644 hearts scored, 855 scored as affected cases (4-severe change, or 5-severe change flaccid), and 15,060 scored as controls (1-normal, or 2-mild change).

TABLE 1
Phenotype Collection and Heart Scoring

| | | |
|---|---|---|
| Observed Heart Score | Count | Percentage |

| | | | |
|---|---|---|---|
| Normal | 1 | 19809 | 60.46% |
| Mild change | 2 | 8640 | 26.37% |
| Moderate change | 3 | 2957 | 9.03% |
| Severe change | 4 | 812 | 2.48% |
| Severe change flaccid | 5 | 545 | 1.66% |
| | Total | 32763 | |

Hair samples from all individuals were collected upon feedlot entry for the purpose of DNA archiving. For sequences and genetic evaluation, a selection of 5,001 contemporary grouped individuals (from the above 32,763) with data captured from feedlot entry to harvest were genotyped using Low-Pass sequencing platform-Gencove (Table 2). Each individual was genotyped for approximately 60 million SNP markers, of which approximately 12 million exhibit variation in the genotyped population. Minor allele frequency (MAF) was approximated using 100 individuals cut off at 10%, which yielded 9M SNPs. The genome was then divided into 1M equally spaced, non-overlapping bins, and 1 SNP per bin was randomly selected.

TABLE 2
Contemporary grouped individuals for Low-Pass sequencing

| BCHF Score | Count |
|---|---|
| 1 or 2 | 4446 |
| 4 or 5 | 555 |

### BCHF is a Heritable Trait

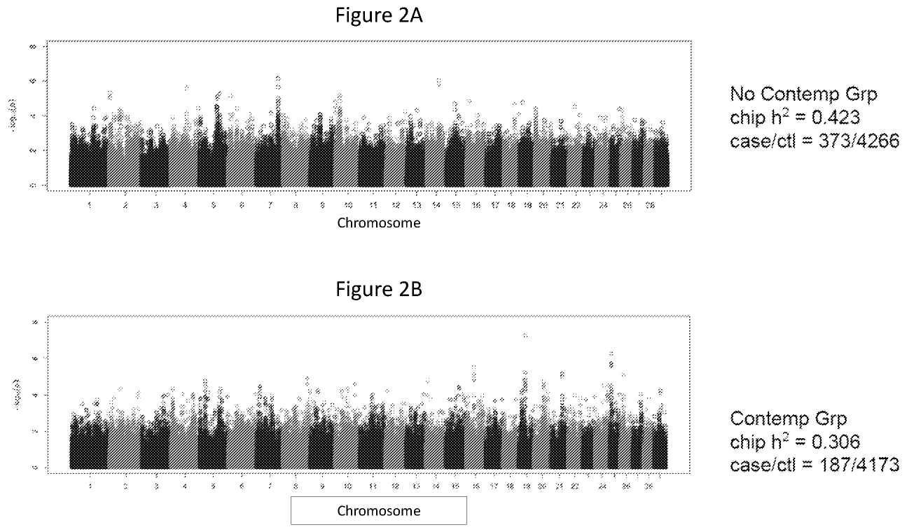

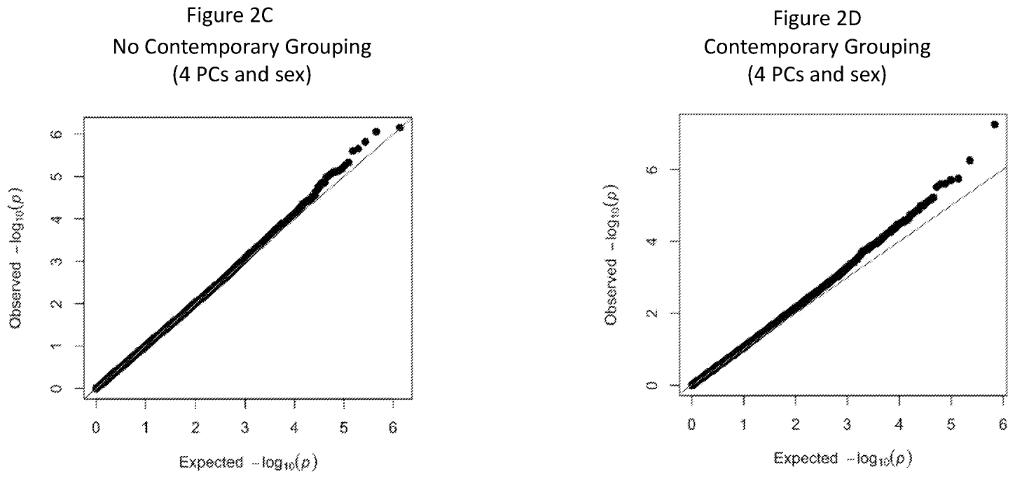

Heritability (h²)—the proportion of trait variance that is due to additive genetic factors, was calculated for both the contemporary and non-contemporary groups (FIGS. 2A-2D). No evidence of single genes having large effects was found, thus BCHF is likely a polygenic trait through QTL. The heritability estimate from a binary threshold-linear Gibbs model was 0.356 (or approximately 35.6%).

Disease Risk is Associated with Angus Ancestry Proportion

Using the genomic best linear unbiased prediction (gBLUP) method, it was found that single chromosomes can capture 70-80% of prediction accuracy. Thus, the BCHF trait is likely associated with population structure, which tends to impact all chromosomes about the same.

TABLE 3
gBLUP - Heart Score Case/Control Population Structure

| | Mean 5-fold | Percent of whole |
|---|---|---|
| Genomic partition | x-val acc* | genome acc* |
| Chromosome 1 | 0.212 | 73.77 |
| Chromosome 2 | 0.218 | 75.87 |
| Chromosome 3 | 0.204 | 71.22 |
| Chromosome 7 | 0.232 | 80.70 |
| Whole genome | 0.287 | — |

*acc = cor(genomic estimated breeding value (GEBV), trait-score), or accuracy

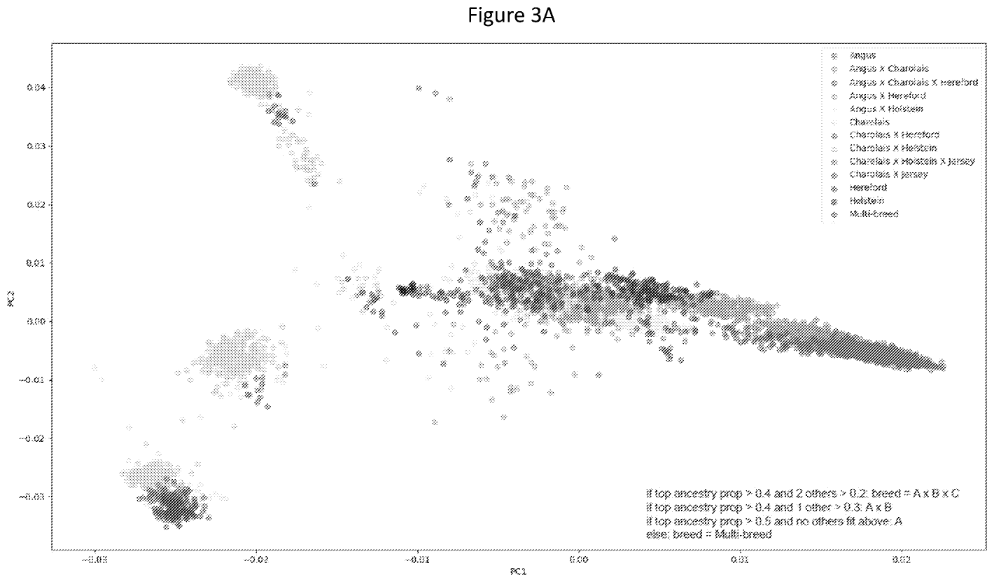

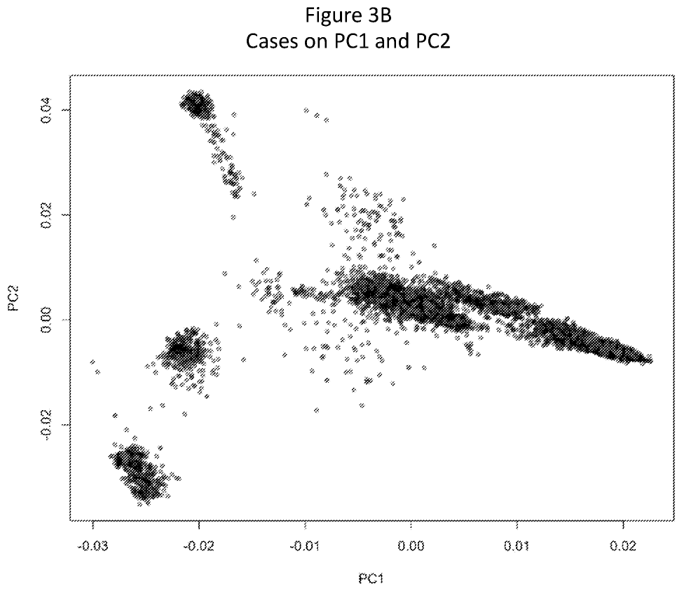

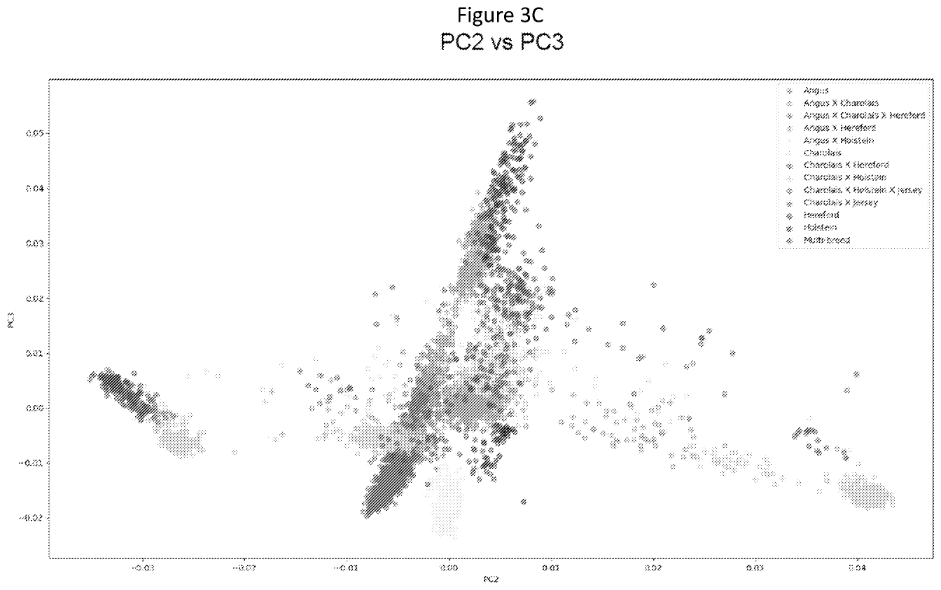

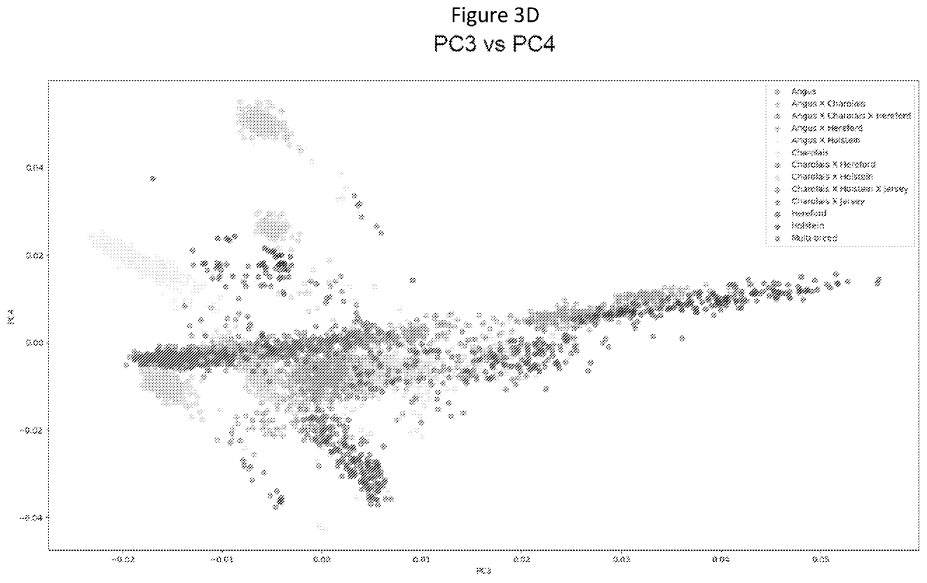

Principle component analysis (PCA) of genomic data was used to further explore differences due to breed in the reference population. Approximately 90 principal component groups were generated, and the first 4 were analyzed (FIGS. 3A-3D).

Following this hypothesis, the observed incidence in specific breeds was analyzed. As shown below in Table 4, cattle having Angus (“Black”) in their pedigree had a higher incidence of BCHF (4.5% had a heart phenotype score of 4 or 5).

TABLE 4
Observed Breed Incidence

| Observed | Heart Score | | | | |
|---|---|---|---|---|---|
| Breed Type | 1 | 2 | 3 | 4 | 5 |
| Angus | 56.71% | 27.70% | 11.09% | 3.31% | 1.19% |
| | (1,335) | (652) | (261) | (78) | (28) |
| Angus × Hereford | 62.85% | 24.94% | 9.18% | 1.92% | 1.11% |
| | (1,308) | (519) | (191) | (40) | (23) |
| Angus × Dairy | 71.83% | 20.56% | 5.96% | 1.21% | 0.43% |
| | (2,012) | (576) | (167) | (34) | (12) |
| Charolais × Dairy | 63.99% | 26.54% | 7.83% | 1.43% | 0.21% |
| | (3,007) | (1,247) | (368) | (67) | (10) |
| Charolais × | 68.05% | 21.92% | 8.54% | 1.20% | 0.22% |
| Angus × Hereford | (2,151) | (693) | (270) | (38) | (9) |

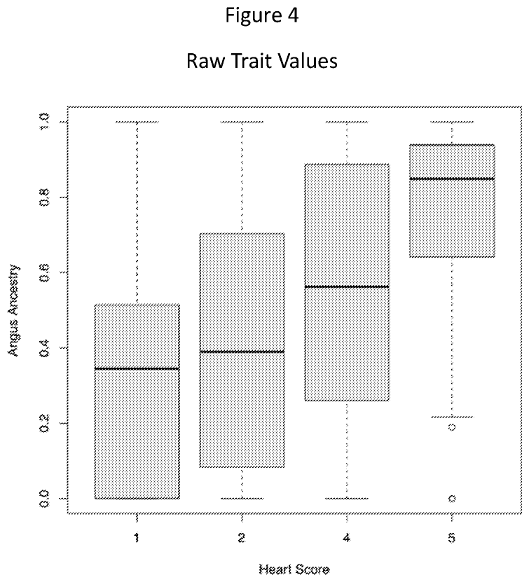

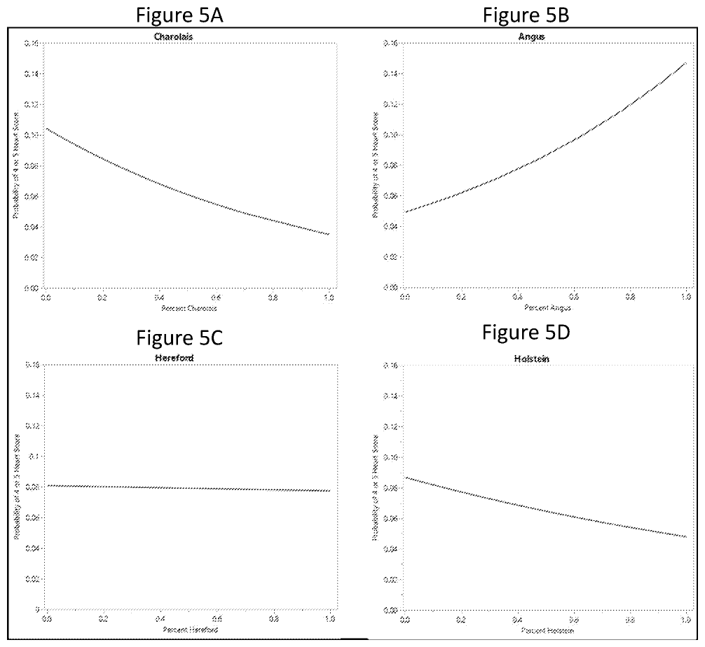

As shown in FIG. 4 and FIG. 5B, the degree of Angus ancestry directly correlated with an increase in BCHF (heart scores of 4 or 5). Using logistic regression, the risk of BCHF is approximately 0.050 (5.0%) with no Angus ancestry (x=0). Some Angus ancestry (x=0.5) increases the risk to 0.080 (8.0%) and for full Angus cattle (x=1) the risk is 0.150 (15.0%).

In some embodiments, the disclosure teaches a method for calculating the probability of a bovine developing congestive heart failure, comprising genotyping at least one bovine using a set of molecular markers and gathering data; analyzing said data to determine a percentage of Angus, Charolais, Hereford, and/or Holstein ancestry; and calculating the probability of the at least one bovine developing congestive heart failure based on ancestry.

### BCHF Genetic Correlation to Carcass and Growth

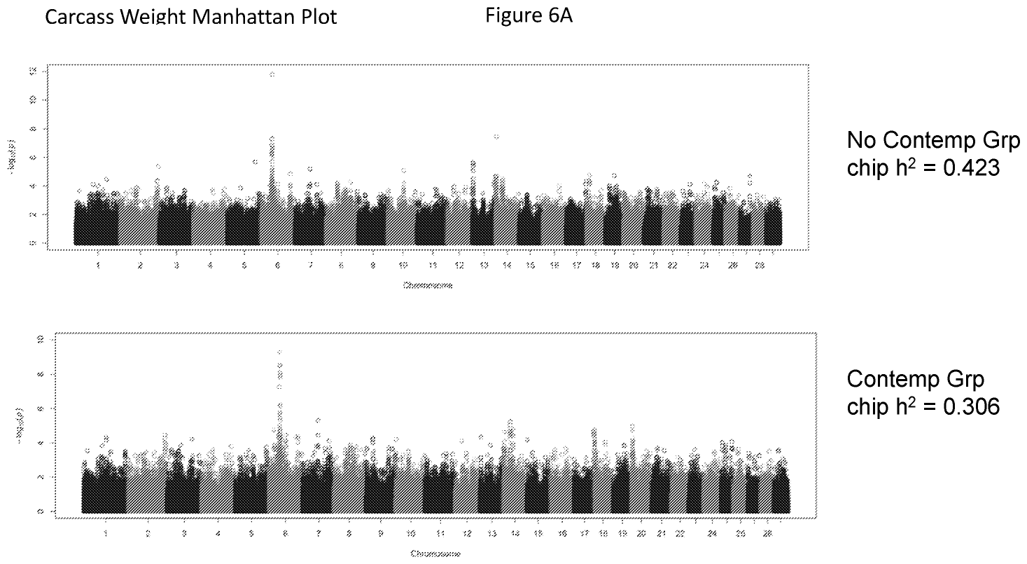

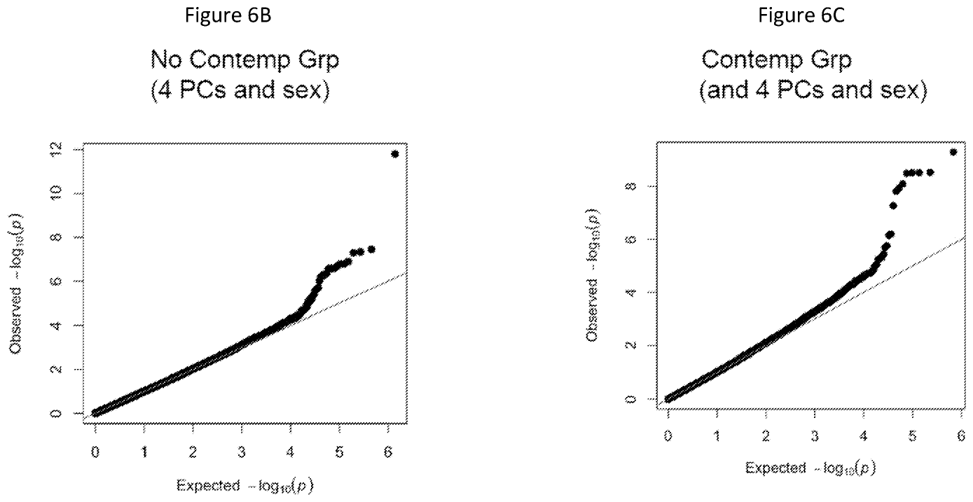

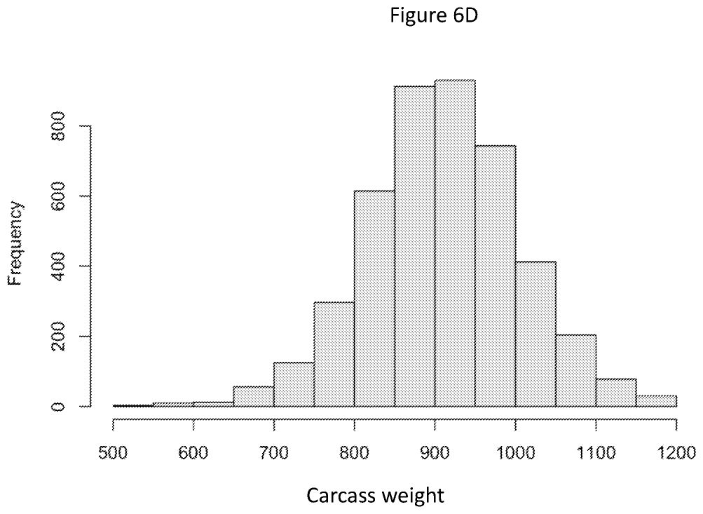

Using the THRGIBBSF90 threshold-linear model (Tsuruta, S. and I. Misztal. 2006. THRGIBBSF90 for estimation of variance components with threshold and linear models. Proc. 8^{th} World Congress Gen. Appl. Livest. Prod., Belo Horizonte, Brazil), correlations with other bovine traits was examined. Generally, a value of greater than 0.2 indicates a moderate positive correlation, with values above 0.4 to be a relatively strong positive correlation. As shown below in Table 5, positive correlations to carcass weight and growth (both average daily gain and dry matter/feed intake) were identified (see also FIGS. 6A-D for Manhattan (FIG. 6A), q-q plots (FIGS. 6B and 6C), and histogram (FIG. 6D) related to carcass weight).

TABLE 5
Genetic correlation to carcass and growth

| | | BCHF |
|---|---|---|
| Trait | Heritability | Correlation |
| BCHF | 0.356 | — |
| Carcass weight, head off and viscera | 0.387 | 0.460 |
| removed | | |
| Backfat thickness at 13^{th} rib | 0.497 | −0.120 |
| Area of ribeye at 13^{th} | 0.299 | 0.080 |
| Marbling score, USDA measured with a | 0.216 | −0.108 |
| camera | | |
| Average daily gain, pounds per day 120 days | 0.248 | 0.289 |
| Dry matter intake, feed intake recording | 0.216 | 0.309 |
| system, pounds of feed eaten per day | | |

### Digital Genotype-Phenotype Library

Using the above genomic and phenotype data, associative BCHF digital libraries were created. In some embodiments, the digital genomic library comprises (a) genetic variations associated with each of a plurality of bovine animals; and (b) a digital phenotype library comprising phenotypic metrics associated with each of a plurality of bovine animals.

In some embodiments, the genetic variations are SNPs. In some aspects, the library comprises at least 10,000 SNPs, at least 30,000 SNPs, at least 40,000 SNPs, at least 50,000 SNPs, at least 100,000 SNPs, at least 200,000 SNPs, at least 300,000 SNPs, at least 400,000 SNPs, at least 500,000 SNPs, at least 600,000 SNPs, at least 700,000 SNPs, at least 800,000 SNPs, at least 900,000 SNPs, or at least 1 million SNPs.

In some embodiments, the phenotypic metrics comprise at least one visual physiologic indication of bovine heart disease. In some aspects, the phenotypic metrics comprise at least one heart score indicative of bovine heart disease.

In some embodiments, the plurality of bovine animals in the digital genomic and phenotype library is at least 1000 bovines. In some aspects, the plurality of bovine animals in the digital genomic and phenotype library is at least 2,000 bovines, at least 3,000 bovines, at least 4,000 bovines, at least 5,000 bovines, at least 6,000 bovines, at least 7,000 bovines, at least 8,000 bovines, at least 9,000 bovines, or at least 10,000 bovines.

In some embodiments, the disclosure relates to a processor-readable non-transitory medium storing code representing instructions to be executed by a processor, the code comprising code to cause the processor to: (a) access data from a digital genomic library comprising genetic variations associated with each of a plurality of bovine animals; (b) access data from a digital phenotype library comprising phenotypic metrics associated with each of a plurality of bovine animals; (c) determine, utilizing a genomic BLUP model, a correlation between the genetic variations present in the digital genomic library and a phenotypic metric present in the digital phenotype library. In some aspects, the code includes code to cause the processor to: (d) produce an algorithm utilizing the correlation from step (c) that has a function of calculating an EPD for a bovine animal based upon genomic data for said animal.

In some embodiments, the disclosure teaches a method for generating an algorithm to predict a phenotype in a bovine animal, comprising: sequencing the genomes of a plurality of bovine animals to obtain sequence data; phenotyping the plurality of bovine animals post-mortem to obtain phenotypic data, using genomic BLUP to correlate the sequencing data with at least one phenotype of interest from the phenotypic data; and producing an algorithm to predict the at least one phenotype of interest in a bovine animal. In some aspects, the phenotype is heart disease. In some aspects, the heart disease is BCHF. In some aspects, the plurality of bovine animals are Bos taurus. In some aspects, use of the algorithm yields an EPD.

### EPD

EPDs are increasingly being used to select sires (or bulls) and cows and heifers (or egg donors) as they can provide a more accurate prediction of phenotypes of progeny, compared to selection of a sire or dam based on individual performance. EPD values for specific traits are calculated based on data from the individual, their progeny, their relatives, and DNA analysis. The accuracy of an EPD thus translates to confidence in a prediction of the animals expected performance. Accurate EPDs allow animal owners to make informed decisions about breeding pairs.

In some embodiments, the disclosure relates to an EPD that predicts the risk of congestive heart failure in a progeny of a bovine animal. In some embodiments, the EPD has an accuracy of greater than 50%. In some embodiments, the EPD has an accuracy of greater than 60%. In some embodiments, the EPD has an accuracy of greater than 70%. In some embodiments, the EPD has an accuracy of greater than 80%. In some embodiments, the EPD has an accuracy of greater than 90%.

In some embodiments, the EPDs generated by the methods disclosed herein may be compiled with other animal records by reputable organizations, such as the American Angus Association. These records include EPD charts for individual animals and breed percentile EPD charts.

In some embodiments, the disclosure teaches a method for producing an EPD that predicts the risk of BCHF in a progeny of at least one bovine animal, comprising obtaining genomic sequencing data from the at least one bovine animal; analyzing the sequencing data in conjunction with a phenotype-genotype library, wherein the phenotype-genotype library comprises a plurality of phenotypic heart and genomic variation data; and producing a BCHF EPD for the at least one bovine animal.

In some embodiments, the disclosure teaches a method of producing an EPD algorithm, comprising: utilizing a genomic BLUP model to determine a correlation between the genetic variations present in the digital genomic library and a phenotypic metric present in the digital phenotype library to thereby produce an algorithm that calculates an EPD.

In some embodiments, the disclosure teaches a method of predicting the risk of BCHF in progeny of a bovine, comprising: performing a genetic test upon a sire or dam to produce genomic data; and utilizing an algorithm as taught herein to analyze the genomic data and return an EPD.

In some embodiments, the EPD is generated from an analysis of a high-density genomic test. In some aspects, the sequencing data is obtained from a high-density bovine SNP array, wherein the array comprises SNPs associated with genetic risk factors for bovine congestive heart failure.

In some aspects, the EPD is generated from an analysis of an existing genotype database. Examples of bovine databases include, but are not limited to, Bovine Genome Database (available on the world wide web at BovineGenome.org), Bovine Genome Variation Database (available on the world wide web at animal.nwsuaf.edu.cn/BosVar), Cattle Genome Analysis Data Repository (available on the world wide web at animalgenome.org/repository/cattle/), CattleQTLdb (available on the world wide web at animalgenome.org/cgi-bin/QTLdb/BT/index), and Cattle SNP database (available on the world wide web at ncbi.nlm.nih.gov/SNP/snp_batchSearch.cgi?org-9913&type=SNP).

Breeding with EPDs

Selective mating, or selective breeding, is based on identifying the best progeny from one generation and making them the parents for the next generation. Selective breeding thus relies on accurate phenotypic and/or genetic information to make the correct decisions regarding which individuals to breed with pass on superior traits. As described above, EPD values for specific traits are calculated based on data from the individual, their progeny, their relatives, and DNA analysis. An accurate EPD thus translates to confidence in a prediction of a particular trait being passed on to the next generation.

In some embodiments, the EPDs generated by the methods disclosed herein are used to make breeding decisions. In some embodiments, the disclosure teaches a method for producing a bovine progeny, comprising selecting at least one male bovine having EPDs criteria associated therewith, wherein the EPD criteria includes an EPD for BCHF, and wherein the BCHF EPD is less than 0.1; pairing the selected male with at least one female; and producing at least one bovine progeny. In some aspects, the at least one male bovine has a BCHF EPD of less than 0.0. In some aspects, the at least one male bovine has a BCHF EPD of less than-0.1. In some aspects, the at least one male bovine has a BCHF EPD of less than-0.2. In some aspects, the at least one male bovine has a BCHF EPD of less than-0.02.

In some embodiments, the pairing of the selected male with at least one female comprises: selecting at least one female bovine having EPDs criteria associated therewith, wherein the EPD criteria includes an EPD for BCHF; calculating the average combined EPD criteria for each male-female combination; and selecting a male-female combination to produce a mating pair, wherein the average combined EPD criteria for BCHF in said mating pair is less than 0.1. In some aspects, the average combined EPD criteria for BCHF in said mating pair is less than 0.0. In some aspects, the average combined EPD criteria for BCHF in said mating pair is less than −0.1. In some aspects, the average combined EPD criteria for BCHF in said mating pair is less than −0.2. In some aspects, the average combined EPD criteria for BCHF in said mating pair is less than −0.02.

In some aspects, the at least one bovine progeny has a less than 25% chance of developing BCHF. In some aspects, the at least one bovine progeny has a less than 24% chance, less than 23% chance, less than 22% chance, less than 21% chance, less than 20% chance, less than 19% chance, less than 18% chance, less than 17% chance, less than 16% chance, less than 15% chance, less than 14% chance, less than 13% chance, less than 12% chance, less than 11% chance, less than 10% chance, less than 9% chance, less than 8% chance, less than 7% chance, less than 6% chance, less than 5% chance, less than 4% chance, less than 3% chance, less than 2% chance, or less than 1% chance of developing BCHF.

In some embodiments, the methods disclosed herein reduce the genetic risk factors for BCHF in a bovine population. In some embodiments, the methods disclosed herein reduce the incidence of BCHF in a bovine population. In some aspects, the incidence of BCHF in a bovine population is reduced to less than 10%, less than 9%, less than 8%, less than 7%, less than 6%, less than 5%, less than 4%, less than 3%, less than 2%, less than 1%.

In some embodiments, the methods disclosed herein increases the genetic merit of at least one bovine progeny. In some aspects, the increased genetic merit is an EPD for BCHF that is less than 0.1. In some aspects, the at least one bovine progeny has a BCHF EPD of less than 0.0. In some aspects, the at least one bovine progeny has a BCHF EPD of less than −0.1. In some aspects, the at least one bovine progeny has a BCHF EPD of less than −0.2. In some aspects, the at least one bovine progeny has a BCHF EPD of less than −0.02.

### Genetic Kits

There are currently breed specific genetic tests on the market, for example, Angus-GS by Neogen GeneSeek (available on the world wide web at angus.org/AGI/angusGS).

Thus, in some embodiments, the disclosure relates to a kit comprising a bovine breed specific genetic test, a bovine congestive heart failure genetic test, and a genotype-phenotype library.

In some aspects, the bovine breed specific genetic test is a high-density SNP array. In some aspects, the bovine congestive heart failure genetic test is a high-density SNP array.

### Breeds of Cattle

The methods, genetic tests, digital libraries, and EPDs generated from said tests are applicable to any breed or species of bovine.

Exemplary cattle breeds include, but are not limited to, Angus, or Aberdeen-Angus, Ayrshire, Beefmaster, Belgium Blue, Belted Galloway, Brahman, or Zebu, Brangus, British White, Brown Swiss, Charolais, Chianina, Devon, Dexter, English Longhorn, Galloway, Gloucester, Guernsey, Hereford, or Whiteface, Highland, Holstein-Friesian, Irish Moiled, Jersey, Kerry, Limousin, Luing, Milking Devon, Milking Shorthorn, Normande, Polled Hereford, Red Angus, Red Poll, Santa Gertrudis, Shorthorn, or Durham, South Devon, Simmental, Sussex, Welsh Black, and White Park.

### EXAMPLES

### Example 1: EPDs Generated with the Methods Disclosed Herein have Greater than 70% Accuracy

An analysis to associate Low-Pass genotype data with phenotypes on the selected population was carried out at Gencove, Inc. The normal and case phenotype definitions were fitted in a binary genomic BLUP model (best linear unbiased prediction) with the first four principal components of the genomic relationship matrix (GRM) fitted as effects using the EMMAX software package in R (available on the world wide web at genetics.cs.ucla.edu/emmax/). The resulting random animal effects were extracted for each individual and used as the resulting estimated breeding value (EBV). An expected progeny difference (EPD) was calculated for each individual as 0.5*EBV.

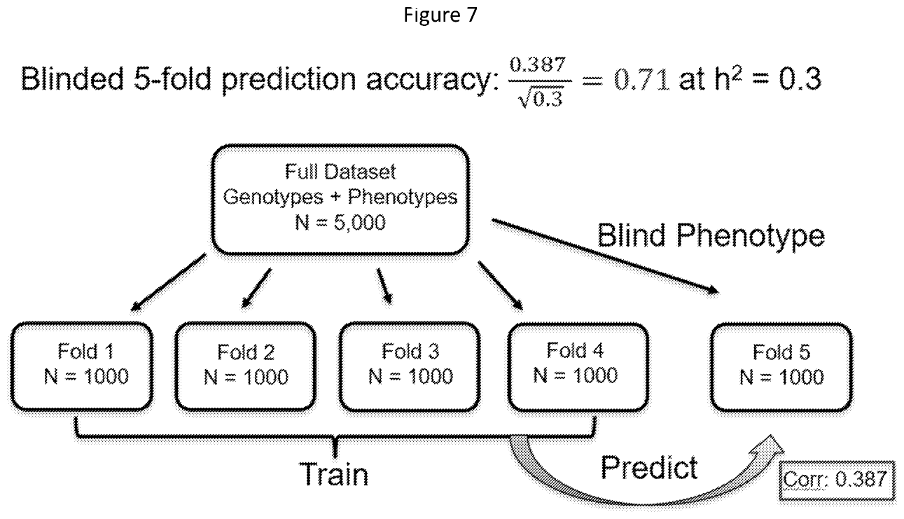

The resulting EPD prediction was validated using a blinded 5-fold model. The correlation between the blinded prediction and the phenotype was 0.387. The EPD accuracy of 0.71 was calculated as the blinded 5-fold prediction correlation divided by the square root of the trait heritability (0.3) (FIG. 7).

The final dataset and EPD model can be used to provide predictions on new individuals (a genetic analysis system) added to the model by providing a genotype with which a new EPD can be calculated (blinded because that individual would not have a phenotype in the model). The expected accuracy of that blind prediction is 0.71. The EPD accuracy of the model can be increased by adding both phenotyped and genotyped individuals to the training dataset.

### Example 2: Using the BCHF EPD as a Breeding Tool to Reduce Costs Associated with Death from Congestive Heart Failure

Based on the above accuracy of the EPDs generated by the methods and libraries disclosed herein, it was hypothesized that this would be a cost-effective method for identifying cattle that are likely to produce progeny that are at risk for BCHF. Economic models to estimate the value of reducing the incidence of BCHF cases in feedlots were generated.

Profit and loss (P/L) were determined by difference in death loss from heart cases with all other factors held constant. Dead cost incurred through 217 DOF (average DOF for heart case deaths). There were no assumptions of management strategy (shipping fragiles). The analysis of a low incidence scenario is presented below in Tables 6A-6C.

TABLE 6A
P/L analysis from weaning to harvest
at low incidence death loss scenario

| Death loss | 0.60% | Death loss | 0.20% |
|---|---|---|---|
| Score 1% | 56.9% | Score 1% | 85.6% |
| Score 2% | 22.0% | Score 2% | 7.3% |
| Score 3% | 13.7% | Score 3% | 4.6% |
| Score 4% | 4.1% | Score 4% | 1.4% |
| Score 5% | 3.3% | Score 5% | 1.1% |
| Sum | 100.0% | Sum | 100.00 |

TABLE 6B
P/L analysis from weaning to harvest
at low incidence death loss scenario

| Death loss | P/L per HD |
|---|---|
| 0.20% | 3.65 |
| 0.60% | 10.98 |
| Difference | $7.33 |

TABLE 6C
P/L analysis from weaning to harvest
at low incidence death loss scenario

| | |
|---|---|
| # of calves/yr/CM bull | 15 |
| # of breeding seasons | 4 |
| Cut death loss in half | $7.33 |
| Value per bull | $439.80 |

A higher incidence scenario is shown below in Tables 7A-7C.

TABLE 7A
P/L analysis from weaning to harvest
at low incidence death loss scenario

| Death loss | 0.50% | Death loss | 1.00% |
|---|---|---|---|
| Score 1% | 64.1% | Score 1% | 28.1% |
| Score 2% | 18.3% | Score 2% | 36.6% |
| Score 3% | 11.4% | Score 3% | 22.9% |
| Score 4% | 3.4% | Score 4% | 6.9% |
| Score 5% | 2.8% | Score 5% | 5.5% |
| Sum | 100.0% | Sum | 100.00 |

TABLE 7B
P/L analysis from weaning to harvest
at low incidence death loss scenario

| Death loss | P/L per HD |
|---|---|
| 0.50% | 9.14 |
| 1.00% | 18.33 |
| Difference | $9.19 |

TABLE 7C
P/L analysis from weaning to harvest
at low incidence death loss scenario

| | |
|---|---|
| # of calves/yr/CM bull | 15 |
| # of breeding seasons | 4 |
| Cut death loss in half | $9.19 |
| Value per bull | $551.40 |

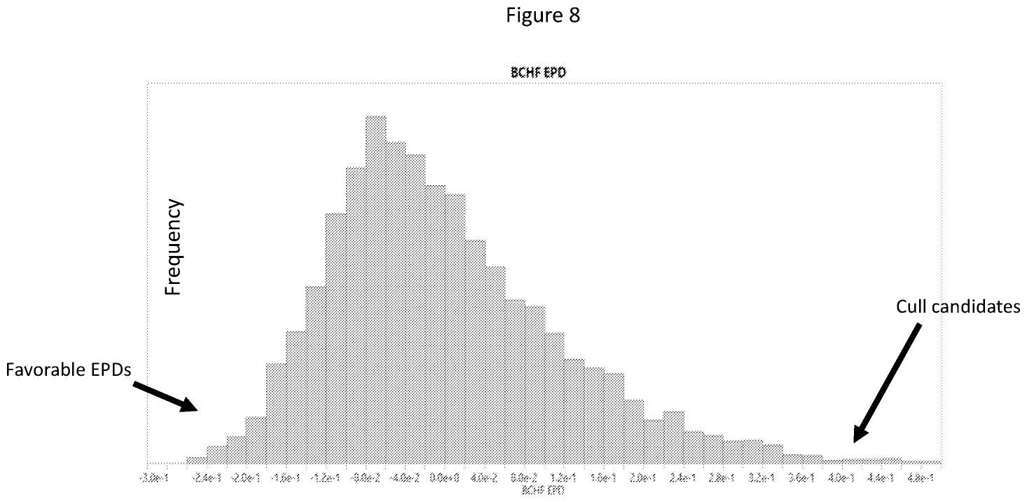

Thus, if the cost of the test is approximately $20, testing the males prior to breeding with them could save $430 or more (see also FIG. 8 for example favorable EPDs for breeding).

### Example 3: Using EPDs for Individual Animal Prediction and Management

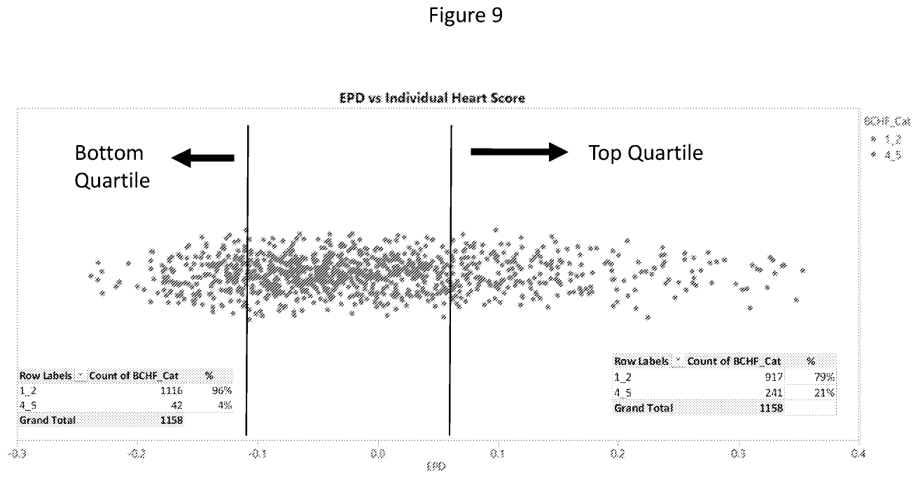

In addition to predicting the performance of the progeny, the EPDs generated as described herein may also predict the performance of the individual animal. As shown in FIG. 9, 96% of those in the lower quartile of EPDs had a heart score of 1 or 2, whereas 21% of those in the top quartile of EPDs had a heart score of 4 or 5. Thus, animals with EPDs of greater than, 0.2, for example, not only have a much higher chance of having progeny with BCHF, but are also at much greater risk for developing BCHF themselves.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### Numbered Embodiments

1. An expected progeny difference (EPD) that predicts the risk of congestive heart failure in a progeny of a bovine animal, wherein said EPD has an accuracy of greater than 70%.
2. The expected progeny difference of embodiment 1, wherein the bovine animal is Bos taurus.
3. The expected progeny difference of embodiment 1 or 2, wherein the bovine animal is selected from one of the following breeds: Angus, Ayrshire, Beefmaster, Belgium Blue, Belted Galloway, Brahman, Brangus, British White, Brown Swiss, Charolais, Chianina, Devon, Dexter, English Longhorn, Galloway, Gloucester, Guernsey, Hereford, Highland, Holstein-Friesian, Irish Moiled, Jersey, Kerry, Limousin, Luing, Milking Devon, Milking Shorthorn, Normande, Polled Hereford, Red Angus, Red Poll, Santa Gertrudis, Shorthorn, South Devon, Simmental, Sussex, Welsh Black, and White Park.
4. The expected progeny difference (EPD) of any one of embodiments 1-3, wherein the EPD is generated from an analysis of a high-density genomic test.
5. The expected progeny difference of embodiment 4, wherein the high-density genomic test is a set of at least 1 million SNPs that span the bovine genome.
6. The expected progeny difference of any one of embodiments 4-5, wherein the genetic analysis is performed from an existing genotype database.
7. A method for producing a bovine progeny having a less than 50% chance of developing bovine congestive heart failure (BCHF), comprising:
   selecting at least one male bovine having expected progeny differences (EPDs) criteria associated therewith, wherein the EPD criteria includes an EPD for BCHF, and wherein the BCHF EPD is less than 0.1;
   pairing the selected male with at least one female; and
   producing at least one bovine progeny, wherein the bovine progeny has a less than 50% chance of developing BCHF.
8. The method of embodiment 7, wherein the bovine congestive heart failure expected progeny difference is less than 0.0.
9. The method of embodiment 7, wherein the bovine congestive heart failure expected progeny difference is less than-0.1.
10. The method of embodiment 7, wherein the bovine congestive heart failure expected progeny difference is less than-0.2.
11. The method of embodiment 7, wherein the bovine congestive heart failure expected progeny difference is less than-0.02.
12. The method of embodiment 7, wherein the pairing the selected male with at least one female comprises:
   selecting at least one female bovine having expected progeny differences (EPDs) criteria associated therewith, wherein the EPD criteria includes an EPD for bovine congestive heart failure (BCHF);
   calculating the average combined EPD criteria for each male-female combination; and
   selecting a male-female combination to produce a mating pair, wherein the average combined EPD criteria for BCHF in said mating pair is less than 0.1.
13. The method of embodiment 12, wherein the average combined expected progeny difference criteria for bovine congestive heart failure in said mating pair is less than 0.0.
14. The method of embodiment 12, wherein the average combined expected progeny difference criteria for bovine congestive heart failure is less than-0.1.
15. The method of embodiment 12, wherein the wherein the average combined expected progeny difference criteria for bovine congestive heart failure is less than-0.2.
16. The method of embodiment 12, wherein the wherein the average combined expected progeny difference criteria for bovine congestive heart failure is less than-0.02.
17. The method of any one of embodiments 7-16, wherein the at least one bovine progeny has a less than 25% chance of developing bovine congestive heart failure.
18. The method of any one of embodiments 7-16, wherein the at least one bovine progeny has a less than 20% chance of developing bovine congestive heart failure.
19. The method of any one of embodiments 7-16, wherein the at least one bovine progeny has a less than 15% chance of developing bovine congestive heart failure.
20 The method of any one of embodiments 7-16, wherein the at least one bovine progeny has a less than 10% chance of developing bovine congestive heart failure.
21. The method of any one of embodiments 7-16, wherein the at least one bovine progeny has a less than 5% chance of developing bovine congestive heart failure.
22. The method of any one of embodiments 7-16, wherein the at least one bovine progeny has a less than 1% chance of developing bovine congestive heart failure.
23. The method of any one of embodiments 7-22, wherein the method reduces the genetic risk factors for bovine congestive heart failure in a bovine population.
24 The method of any one of embodiments 7-23, wherein the method reduces the incidence of bovine congestive heart failure in a bovine population.
25. The method of any one of embodiments 7-24, wherein the method reduces the incidence of bovine congestive heart failure in a bovine population to less than 10%.
26 The method of any one of embodiments 7-24, wherein the method reduces the incidence of bovine congestive heart failure in a bovine population to less than 7%
27. The method of any one of embodiments 7-24, wherein the method reduces the incidence of bovine congestive heart failure in a bovine population to less than 5%.
28. The method of any one of embodiments 7-24, wherein the method reduces the incidence of bovine congestive heart failure in a bovine population to less than 3%.
29. The method of any one of embodiments 7-24, wherein the method reduces the incidence of bovine congestive heart failure in a bovine population to less than 1%
30. The method of embodiment 7 or 12, wherein the method increases the genetic merit of the at least one bovine progeny.
31 The method of any one of embodiments 7-30, wherein the bovine animal is Bos taurus.
32. The method of any one of embodiments 7-31, wherein the bovine animal is selected from one of the following breeds: Angus, Ayrshire, Beefmaster, Belgium Blue, Belted Galloway, Brahman, Brangus, British White, Brown Swiss, Charolais, Chianina, Devon, Dexter, English Longhorn, Galloway, Gloucester, Guernsey, Hereford, Highland, Holstein-Friesian, Irish Moiled, Jersey, Kerry, Limousin, Luing, Milking Devon, Milking Shorthorn, Normande, Polled Hereford, Red Angus, Red Poll, Santa Gertrudis, Shorthorn, South Devon, Simmental, Sussex, Welsh Black, and White Park.
33. The method of any one of embodiments 30-32, wherein the increase in genetic merit of the at least one bovine progeny is a favorable bovine congestive heart failure expected progeny difference.
34. The method of embodiment 33, wherein the favorable bovine congestive heart failure expected progeny difference is less than 0.1.
35. A method for producing an expected progeny difference (EPD) that predicts the risk of bovine congestive heart failure (BCHF) in a progeny of at least one bovine animal, comprising:
   obtaining genomic sequencing data from the at least one bovine animal;
   analyzing the sequencing data in conjunction with a phenotype-genotype library, wherein the phenotype-genotype library comprises a plurality of phenotypic heart and genomic variation data; and
   producing a BCHF EPD for the at least one bovine animal.
36. The method of embodiment 35, wherein the sequencing data is obtained from a high-density bovine SNP array, wherein the array comprises SNPs associated with genetic risk factors for bovine congestive heart failure.
37. The method of embodiment 35, wherein the sequencing data is obtained from an existing database.
38. A bovine congestive heart failure expected progeny difference produced by the method of any one of embodiments 35-37.
39 The bovine congestive heart failure (BCHF) expected progeny difference (EPD) of embodiment 38, wherein the BCHF EPD has an accuracy of at least 70%.
40. A method for generating an algorithm to predict a phenotype in a bovine animal, comprising:
   sequencing the genomes of a plurality of bovine animals to obtain sequence data;
   phenotyping the plurality of bovine animals post-mortem to obtain phenotypic data,
   using genomic BLUP to correlate the sequencing data with at least one phenotype of interest from the phenotypic data; and
   producing an algorithm to predict the at least one phenotype of interest in a bovine animal.
41. The method of embodiment 40, wherein the phenotype is heart disease.
42. The method of embodiment 41, wherein the heart disease is bovine congestive heart failure.
43. The method of any one of embodiments 40-42, wherein the plurality of bovine animals are Bos taurus.
44. The method of embodiment 43, wherein the bovine animal is selected from one of the following breeds: Angus, Ayrshire, Beefmaster, Belgium Blue, Belted Galloway, Brahman, Brangus, British White, Brown Swiss, Charolais, Chianina, Devon, Dexter, English Longhorn, Galloway, Gloucester, Guernsey, Hereford, Highland, Holstein-Friesian, Irish Moiled, Jersey, Kerry, Limousin, Luing, Milking Devon, Milking Shorthorn, Normande, Polled Hereford, Red Angus, Red Poll, Santa Gertrudis, Shorthorn, South Devon, Simmental, Sussex, Welsh Black, and White Park.
45. The method of any one of embodiments 40-44, wherein use of the algorithm yields an expected progeny difference.
46. The method of embodiment 45, wherein the expected progeny difference has an accuracy of at least 70%.
47. A kit comprising:
   a bovine breed specific genetic test;
   a bovine congestive heart failure genetic test; and
   a genotype-phenotype library.
48. The kit of embodiment 47, wherein the bovine breed specific genetic test is a high-density SNP array.
49. The kit of embodiment 47 or 48, wherein the bovine congestive heart failure genetic test is a high-density SNP array.
50. The kit of any one of embodiments 47-49, wherein the bovine breed specific genetic test is Angus.
51. A method for calculating the probability of a bovine developing congestive heart failure, comprising:
   genotyping at least one bovine using a set of molecular markers and gathering data;
   analyzing said data to determine a percentage of Angus, Charolais, Hereford, and/or Holstein ancestry; and
   calculating the probability of the at least one bovine developing congestive heart failure based on ancestry.
52. An associative bovine congestive heart failure (BCHF) digital library, comprising:
   (a) a digital genomic library comprising genetic variations associated with each of a plurality of bovine animals; and
   (b) a digital phenotype library comprising phenotypic metrics associated with each of a plurality of bovine animals.
53. The BCHF digital library of embodiment 52, wherein the genetic variations are single nucleotide polymorphisms.
54. The BCHF digital library of embodiment 52, wherein the genetic variations are at least 100,000 single nucleotide polymorphisms.
55. The BCHF digital library of embodiment 52, wherein the genetic variations are at least 1 million single nucleotide polymorphisms.
56. The BCHF digital library of embodiment 52, wherein the phenotypic metrics comprise at least one visual physiologic indication of bovine heart disease.
57. The BCHF digital library of embodiment 52, wherein the phenotypic metrics comprise at least one heart score indicative of bovine heart disease.
58. The BCHF digital library of embodiment 52, wherein the plurality of bovine animals in the digital genomic and phenotype library is at least 1000 bovines.
59. The BCHF digital library of embodiment 52, wherein the plurality of bovine animals in the digital genomic and phenotype library is at least 5000 bovines.
60. The BCHF digital library of embodiment 52, wherein the plurality of bovine animals in the digital phenotype library is at least 10,000 bovines.
61. A method of producing an expected progeny difference (EPD) algorithm for bovine congestive heart failure (BCHF), comprising: utilizing a genomic BLUPmodel to determine a correlation between genetic variations present in a digital genomic library and a phenotypic metric present in a digital phenotype library to thereby produce an algorithm that calculates an EPD for BCHF.
62. The method of embodiment 61, wherein the EPD has an accuracy of at least 50%.
63. The method of embodiment 61, wherein the EPD has an accuracy of at least 60%.
64. The method of embodiment 61, wherein the EPD has an accuracy of at least 70%.
65. A method of predicting the risk of BCHF in progeny of a bovine, comprising: performing a genetic test upon a sire or dam to produce genomic data; and utilizing the algorithm of embodiment 61 to analyze the genomic data and return an EPD.
66. The method of any one of embodiments 35-39, wherein the analyzing the sequence data in conjunction with a phenotype-genotype library comprises utilizing a processor-readable non-transitory medium storing code representing instructions to be executed by a processor, the code comprising code to cause the processor to:
   (a) access data from a digital genomic library comprising genetic variations associated with each of a plurality of bovine animals;
   (b) access data from a digital phenotype library comprising phenotypic metrics associated with each of a plurality of bovine animals;
   (c) determine, utilizing a genomic BLUP model, a correlation between the genetic variations present in the digital genomic library and a phenotypic metric present in the digital phenotype library.
67. The processor-readable non-transitory medium of embodiment 63, wherein the code includes code to cause the processor to:
   (d) produce an algorithm utilizing the correlation from step (c) that has a function of calculating an expected progeny difference (EPD) for a bovine animal based upon genomic data for said animal.

1. A method for producing a bovine progeny having a less than 50% chance of developing bovine congestive heart failure (BCHF), comprising:
selecting at least one male bovine having expected progeny differences (EPDs) criteria associated therewith, wherein the EPD criteria includes an EPD for BCHF, and wherein the BCHF EPD is less than 0.1;
pairing the selected male with at least one female; and
producing at least one bovine progeny, wherein the bovine progeny has a less than 50% chance of developing BCHF.

2. The method of claim 1 wherein the bovine congestive heart failure expected progeny difference is less than 0.0, less than −0.1, less than −0.2, or less than −0.02.

3.-5. (canceled)

6. The method of claim 1, wherein the pairing the selected male with at least one female comprises:
selecting at least one female bovine having expected progeny differences (EPDs) criteria associated therewith, wherein the EPD criteria includes an EPD for bovine congestive heart failure (BCHF);
calculating the average combined EPD criteria for each male-female combination; and
selecting a male-female combination to produce a mating pair, wherein the average combined EPD criteria for BCHF in said mating pair is less than 0.1.

7. The method of claim 6, wherein the average combined expected progeny difference criteria for bovine congestive heart failure in said mating pair is less than 0.0, less than −0.1, less than −0.2, or less than −0.02.

8.-10. (canceled)

11. The method of claim 1, wherein the at least one bovine progeny has a less than 25% chance, less than 20% chance, less than 15% chance, less than 10% chance, less than 5% chance, or less than 1% chance of developing bovine congestive heart failure.

12.-16. (canceled)

17. The method of claim 1, wherein the method reduces the genetic risk factors for bovine congestive heart failure in a bovine population.

18. The method of claim 1, wherein the method reduces the incidence of bovine congestive heart failure in a bovine population.

19. The method of claim 1, wherein the method reduces the incidence of bovine congestive heart failure in a bovine population to less than 10%, less than 7%, less than 5%, less than 3%, or less than 1%.

20.-23. (canceled)

24. The method of claim 1, wherein the method increases the genetic merit of the at least one bovine progeny.

25. The method of claim 1, wherein the bovine animal is Bos taurus.

26. The method of claim 1, wherein the bovine animal is selected from one of the following breeds: Angus, Ayrshire, Beefmaster, Belgium Blue, Belted Galloway, Brahman, Brangus, British White, Brown Swiss, Charolais, Chianina, Devon, Dexter, English Longhorn, Galloway, Gloucester, Guernsey, Hereford, Highland, Holstein-Friesian, Irish Moiled, Jersey, Kerry, Limousin, Luing, Milking Devon, Milking Shorthorn, Normande, Polled Hereford, Red Angus, Red Poll, Santa Gertrudis, Shorthorn, South Devon, Simmental, Sussex, Welsh Black, and White Park.

27. The method of claim 24, wherein the increase in genetic merit of the at least one bovine progeny is a favorable bovine congestive heart failure expected progeny difference.

28. The method of claim 27, wherein the favorable bovine congestive heart failure expected progeny difference is less than 0.1.

29. A method for producing an expected progeny difference (EPD) that predicts the risk of bovine congestive heart failure (BCHF) in a progeny of at least one bovine animal, comprising:
obtaining genomic sequencing data from the at least one bovine animal;
analyzing the sequencing data in conjunction with a phenotype-genotype library, wherein the phenotype-genotype library comprises a plurality of phenotypic heart and genomic variation data; and
producing a BCHF EPD for the at least one bovine animal.

30. The method of claim 29, wherein the sequencing data is obtained from a high-density bovine SNP array, wherein the array comprises SNPs associated with genetic risk factors for bovine congestive heart failure.

31. (canceled)

32. A bovine congestive heart failure (BCHF) expected progeny difference (EPD) produced by the method of claim 29, wherein the BCHF EPD has an accuracy of at least 70%.

33. (canceled)

34. A method of producing an expected progeny difference (EPD) algorithm for bovine congestive heart failure (BCHF), comprising: utilizing a genomic best linear unbiased prediction model to determine a correlation between genetic variations present in a digital genomic library and a phenotypic metric present in a digital phenotype library to thereby produce an algorithm that calculates an EPD for BCHF.

35. The method of claim 34, wherein the EPD has an accuracy of at least 50%, at least 60%, or at least 70%.

36.-37. (canceled)

38. A method of predicting the risk of BCHF in progeny of a bovine, comprising:
performing a genetic test upon a sire or dam to produce genomic data; and
utilizing the algorithm of claim 34 to analyze the genomic data and return an EPD.

39. The method of claim 29, wherein the analyzing the sequencing data in conjunction with a phenotype-genotype library comprises utilizing a processor-readable non-transitory medium storing code representing instructions to be executed by a processor, the code comprising code to cause the processor to:
(a) access data from a digital genomic library comprising genetic variations associated with each of a plurality of bovine animals;
(b) access data from a digital phenotype library comprising phenotypic metrics associated with each of a plurality of bovine animals;
(c) determine, utilizing a genomic best linear unbiased prediction model, a correlation between the genetic variations present in the digital genomic library and a phenotypic metric present in the digital phenotype library.

40. (canceled)
